(19) United States
(12) Patent Application Publication
(10) Pub. No.: US 2018/0353450 A1
(43) Pub. Date: Dec. 13, 2018

(54) METHODS AND COMPOSITIONS FOR TREATING DEMYELINATING DISEASES USING SOBETIROME OR A SOBETIROME PRODRUG AND A PPAR ACTIVATOR

(71) Applicant: NeuroVia, Inc., San Francisco, CA (US)

(72) Inventor: George Mitchell Grass, IV, Tahoe City, CA (US)

(21) Appl. No.: 16/000,509

(22) Filed: Jun. 5, 2018

Related U.S. Application Data
(60) Provisional application No. 62/517,101, filed on Jun. 8, 2017.

(51) Int. Cl. A61K 31/192; A61P 25/28

(52) U.S. Cl. CPC A61K 31/192; A61K 45/06; A61K 2300/00; A61P 25/28

(57) ABSTRACT

Disclosed are methods for treating a subject having or at risk of developing a disease or condition associated with demyelination, insufficient myelination, or underdevelopment of myelin sheath, by administering an effective amount of sobetirome or a sobetirome prodrug and one or more PPAR activators to the subject. Also disclosed are pharmaceutical compositions in unit dosage form containing an effective amount of sobetirome or a sobetirome prodrug, one or more PPAR activators, and a pharmaceutically acceptable excipient.

### FIELD OF THE INVENTION

The invention relates to methods and compositions for treating demyelinating diseases.

### BACKGROUND

Demyelinating diseases are diseases in which myelin, the lipid substance that forms an insulating sheath around many nerve fibers, is damaged, lost, or exhibits impaired growth or development. As myelination is critical for the rapid conduction of nerve impulses throughout the nervous system, demyelination causes a breakdown in conduction and results in various functional impairments. Subjects suffering from demyelinating diseases may experience muscle weakness, loss of sensation, impaired vision, impaired cognition, ataxia, and paralysis, among other symptoms. Current standards for treating certain demyelinating diseases primarily affect the rate of disease progression. There is a need for new treatments for demyelinating diseases (e.g., for promoting myelination or demyelination).

### SUMMARY OF THE INVENTION

In general, the invention provides methods of administering sobetirome or a sobetirome prodrug and a PPAR activator and compositions therefor.

In one aspect, the invention provides a method of treating a subject having or at risk of developing X-linked adrenoleukodystrophy by administering to the subject an effective amount of sobetirome or a sobetirome prodrug and one or more PPAR activators.

In a related aspect, the invention provides a method of treating a subject having or at risk of developing a disease or condition associated with demyelination, insufficient myelination, or underdevelopment of myelin sheath, comprising administering to the subject an effective amount of sobetirome or a sobetirome prodrug and one or more PPAR activators.

In some embodiments, the disease or condition is multiple sclerosis, a leukodystrophy, a leukoencephalopathy, an idiopathic inflammatory demyelinating disease, or Alzheimer's disease. In certain embodiments, the multiple sclerosis is relapsing-remitting multiple sclerosis, primary-progressive multiple sclerosis, secondary-progressive multiple sclerosis, or progressive-relapsing multiple sclerosis. In particular embodiments, the disease or condition is central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barré syndrome, infantile Refsum disease, adult Refsum disease 1, adult Refsum disease 2, Zellweger syndrome, X-linked adrenoleukodystrophy (X-ALD), metachromatic leukodystrophy, Krabbe disease, Pelizaeus-Merzbacher disease, Canavan disease, Alexander disease, peroneal muscular atrophy, cerebrotendineous xanthomatosis, Binswanger's disease, leukoencephalopathy with vanishing white matter, toxic leukoencephalopathy, van der Knaap disease, progressive multifocal leukoencephalopathy, Marchiafava-Bignami disease or transverse myelitis. In further embodiments, the Guillain-Barré syndrome is acute inflammatory demyelinating polyneuropathy. In yet further embodiments, the chronic inflammatory demyelinating polyneuropathy is multifocal acquired demyelinating sensory and motor neuropathy. In still further embodiments, the chronic inflammatory demyelinating polyneuropathy is induced by HIV infection. In other embodiments, the X-linked adrenoleukodystrophy is adrenomyeloneuropathy or childhood cerebral adrenoleukodystrophy. In yet other embodiments, the X-linked adrenoleukodystrophy is Addison's disease. In still other embodiments, the disease or condition is a chronic axonal neuropathy. In certain embodiments, the disease or condition results from intraventricular hemorrhage, neonatal hypoxia, or acute hypoxemic respiratory failure. In some embodiments, the disease or condition is cerebral palsy. In particular embodiments, administration of sobetirome or the sobetirome prodrug and one or more PPAR activators prevents or mitigates at least one symptom of the disease or condition. In further embodiments, the symptom is a lack of sphincter control, erectile dysfunction, paraparesis, ataxia, adrenocortical insufficiency, progressive neuropathy, paresthesia, dysarthria, dysphagia, clonus, or any combination thereof.

In yet further embodiments, administration of sobetirome or the sobetirome prodrug and one or more PPAR activators prevents or mitigates damage to central nervous system myelin, peripheral nervous system myelin, adrenal cortex, testicular Leydig cells, or any combination thereof. In still further embodiments, each of the one or more PPAR activators is independently a PPARγ agonist or a dual PPARγ and PPARγ agonist. In particular embodiments, the PPARγ agonist is pioglitazone, rosiglitazone, lobeglitazone, ciglitazone, darglitazone, englitazone, netoglitazone, rivoglitazone, or 5-[4-[2-(5-(1-hydroxyethyl)-2-pyridinyl)ethoxy]benzyl]-2,4-thiazolidinedione, or a pharmaceutically acceptable salt thereof. In further embodiments, the PPARγ agonist is pioglitazone or a pharmaceutically acceptable salt thereof. In yet further embodiments, the PPARγ agonist is rosiglitazone or a pharmaceutically acceptable salt thereof. In still further embodiments, the PPARγ agonist is lobeglitazone or a pharmaceutically acceptable salt thereof. In some embodiments, the PPARγ agonist is ciglitazone or a pharmaceutically acceptable salt thereof. In particular embodiments, the PPARγ agonist is darglitazone or a pharmaceutically acceptable salt thereof. In certain embodiments, the PPARγ agonist is englitazone or a pharmaceutically acceptable salt thereof. In other embodiments, the PPARγ agonist is netoglitazone or a pharmaceutically acceptable salt thereof. In yet other embodiments, the PPARγ agonist is rivoglitazone or a pharmaceutically acceptable salt thereof. In still other embodiments, the PPARγ agonist is 5-[4-[2-(5-(1-hydroxyethyl)-2-pyridinyl)ethoxy]benzyl]-2,4-thiazolidinedione or a pharmaceutically acceptable salt thereof. In certain embodiments, the dual PPARγ and PPARγ agonist is aleglitazar, muraglitazar, tesaglitazar, or saroglitazar, or a pharmaceutically acceptable salt thereof. In particular embodiments, the dual PPARγ and PPARγ agonist is aleglitazar or a pharmaceutically acceptable salt thereof. In some embodiments, the dual PPARγ and PPARγ agonist is muraglitazar or a pharmaceutically acceptable salt thereof. In further embodiments, the dual PPARγ and PPARγ agonist is tesaglitazar or a pharmaceutically acceptable salt thereof. In yet further embodiments, the dual PPARγ and PPARγ agonist is saroglitazar or a pharmaceutically acceptable salt thereof.

In still further embodiments, sobetirome or the sobetirome prodrug is administered orally, parenterally, or topically. In certain embodiments, sobetirome or the sobetirome prodrug is administered orally. In particular embodiments, sobetirome or the sobetirome prodrug is administered parenterally. In some embodiments, sobetirome or the sobetirome prodrug is administered buccally, sublingually, sublabially, or by inhalation. In other embodiments, sobetirome or the sobetirome prodrug is administered sublingually. In yet other embodiments, sobetirome or the sobetirome prodrug is administered intramuscularly, intravenously, or subcutaneously. In still other embodiments, sobetirome or the sobetirome prodrug is administered intra-arterially, intravenously, intraventricularly, intramuscularly, subcutaneously, intraspinally, intraorbitally, intrathecally, or intracranially.

In particular embodiments, one or more PPAR activators are administered orally, parenterally, or topically. In certain embodiments, one or more PPAR activators are administered orally. In some embodiments, one or more PPAR activators are administered parenterally. In further embodiments, one or more PPAR activators are administered buccally, sublingually, sublabially, or by inhalation. In yet further embodiments, one or more PPAR activators are administered sublingually. In still further embodiments, one or more PPAR activators are administered intramuscularly, intravenously, or subcutaneously. In other embodiments, one or more PPAR activators is administered intra-arterially, intravenously, intraventricularly, intramuscularly, subcutaneously, intraspinally, intraorbitally, intrathecally, or intracranially.

In yet other embodiments, sobetirome or the sobetirome prodrug is administered in combination with one or more PPAR activators. In still other embodiments, sobetirome or the sobetirome prodrug is administered prior to one or more PPAR activators. In certain embodiments, the sobetirome or sobetirome prodgrug is administered after one or more PPAR activators. In particular embodiments, administration of sobetirome or the sobetirome prodrug and administration of one or more PPAR activators occur within one hour. In some embodiments, administration of sobetirome or the sobetirome prodrug and administration of one or more PPAR activators occur within 12 hours. In further embodiments, administration of sobetirome or the sobetirome prodrug and administration of one or more PPAR activators occur within 24 hours. In yet further embodiments, administration of sobetirome or the sobetirome prodrug and administration of one or more PPAR activators occur within one week. In still further embodiments, administration of sobetirome or the sobetirome prodrug and administration of one or more PPAR activators occur within one month.

In some embodiments, sobetirome or the sobetirome prodrug is administered daily. In certain embodiments, about 1 μg to about 1 mg of sobetirome or the sobetirome prodrug are administered. In particular embodiments, at least 10 μg (e.g., at least 30 μg, at least 50 μg, at least 70 μg, at least 100 μg, or at least 200 μg) of sobetirome or the sobetirome prodrug are administered. In further embodiments, 500 μg or less (e.g., 400 μg or less, 200 μg or less, 100 μg or less, or 70 μg or less) of sobetirome or the sobetirome prodrug are administered. In yet further embodiments, sobetirome is administered. In still further embodiments, the sobetirome prodrug is administered.

In certain embodiments, the PPAR activator is administered at a dose of about 0.1 mg to about 600 mg daily (e.g., about 2 mg to about 200 mg daily).

In a further aspect, the invention provides a pharmaceutical composition comprising sobetirome or a sobetirome prodrug, one or more PPAR activators, and a pharmaceutically acceptable excipient.

In some embodiments, each of the one or more PPAR activators is independently a PPARγ agonist or a dual PPARα and PPARγ agonist. In certain embodiments, each of the one or more PPAR activators is the PPARγ agonist independently selected from the group consisting of pioglitazone, rosiglitazone, lobeglitazone, ciglitazone, darglitazone, englitazone, netoglitazone, rivoglitazone, and 5-[4-[2-(5-(1-hydroxyethyl)-2-pyridinyl)ethoxy]benzyl]-2,4-thiazolidinedione, and pharmaceutically acceptable salts thereof. In particular embodiments, the PPARγ agonist is pioglitazone or a pharmaceutically acceptable salt thereof. In further embodiments, the PPARγ agonist is rosiglitazone or a pharmaceutically acceptable salt thereof. In yet further embodiments, the PPARγ agonist is lobeglitazone or a pharmaceutically acceptable salt thereof. In still further embodiments, the PPARγ agonist is ciglitazone or a pharmaceutically acceptable salt thereof. In some embodiments, the PPARγ agonist is darglitazone or a pharmaceutically acceptable salt thereof. In certain embodiments, the PPARγ agonist is englitazone or a pharmaceutically acceptable salt thereof. In particular embodiments, the PPARγ agonist is netoglitazone or a pharmaceutically acceptable salt thereof. In other embodiments, the PPARγ agonist is rivoglitazone or a pharmaceutically acceptable salt thereof. In yet other embodiments, the PPARγ agonist is 5-[4-[2-(5-(1-hydroxyethyl)-2-pyridinyl)ethoxy]benzyl]-2,4-thiazolidinedione or a pharmaceutically acceptable salt thereof.

In still other embodiments, each of the one or more PPAR activators is independently a dual PPARα and PPARγ agonist selected from the group consisting of aleglitazar, muraglitazar, tesaglitazar, and saroglitazar, and pharmaceutically acceptable salts thereof. In particular embodiments, the dual PPARα and PPARγ agonist is aleglitazar or a pharmaceutically acceptable salt thereof. In certain embodiments, the dual PPARα and PPARγ agonist is muraglitazar or a pharmaceutically acceptable salt thereof. In some embodiments, the dual PPARα and PPARγ agonist is tesaglitazar or a pharmaceutically acceptable salt thereof. In further embodiments, the dual PPARα and PPARγ agonist is saroglitazar or a pharmaceutically acceptable salt thereof. In yet further embodiments, the pharmaceutical composition comprises about 0.1 mg to about 600 mg of the PPAR activator. In still further embodiments, the pharmaceutical composition comprises about 2 mg to about 200 mg of the PPAR activator.

In certain embodiments, the pharmaceutical composition comprises about 1 μg to about 1 mg of sobetirome or the sobetirome prodrug. In particular embodiments, the pharmaceutical composition comprises at least 10 μg (e.g., at least 30 μg, at least 50 μg, at least 70 μg, at least 100 μg, or at least 200 μg) of sobetirome or the sobetirome prodrug. In some embodiments, the pharmaceutical composition comprises 500 μg or less (e.g., 400 μg or less, 200 μg or less, 100 μg or less, 70 μg or less) of sobetirome or the sobetirome prodrug. In further embodiments, the pharmaceutical composition comprises sobetirome. In yet further embodiments, the pharmaceutical composition comprises the sobetirome prodrug. In still further embodiments, the composition is formulated as a unit dosage form selected from the group consisting of a capsule, tablet, troche, film, solution, depot, powder, lozenge, sachet, cachet, elixir, emulsion, and syrup.

### Definitions

“About,” as used herein, refers to a quantity that is ±10% of the indicated value.

“Administration,” as used herein, refers to providing or giving a subject a therapeutic agent (e.g., sobetirome, a sobetirome prodrug, and/or one or more PPAR activators), by any effective route. Exemplary routes of administration are described herein below. Agents may be administered in combination or sequentially.

“Alkenyl,” as used herein, refers to a branched or unbranched, cyclic or acyclic hydrocarbon group containing one or two carbon-carbon double bonds. Alkenyl contains from 2 to 24 carbon atoms. Preferably, alkenyl is lower alkenyl. A lower alkenyl group contains from 2 to 6 carbon atoms (C₂₋₆alkenyl). Preferably, alkenyl is acyclic alkenyl (e.g., acyclic lower alkenyl). Alkenyl may be optionally substituted as described herein.

“Alkyl”, as used herein, refers to a branched or unbranched, cyclic or acyclic saturated hydrocarbon group containing from 1 to 24 carbon atoms. Non-limiting examples of alkyl include methyl, ethyl, n-propyl, isopropyl, n-butyl, isobutyl, t-butyl, pentyl, hexyl, heptyl, octyl, nonyl, decyl, tetradecyl, hexadecyl, eicosyl, tetracosyl and the like. Preferably, alkyl is lower alkyl. A lower alkyl group is a saturated branched or unbranched hydrocarbon having from 1 to 6 carbon atoms (C₁₋₆alkyl). Preferably, alkyl is acyclic alkyl (e.g., acyclic lower alkyl). Alkyl may be optionally substituted as described herein.

“Alkylamino,” as used herein, refers to a heteroalkyl containing one, two, or three nitrogen atoms. An alkylamino can be a straight chain, branched or cycloalkylamino. An alkylamino generally has the structure —NX¹X² or —(NX¹X²X³)⁺ in which X¹, X², and X³ are each independently H, a substituted alkyl, or an unsubstituted alkyl, provided that the group does not have the structure —NH₂ or —NH₃⁺ and the total number of non-hydrogen atoms does not exceed 24. Examples of alkylamino groups include the following structures: —NHCH₃, —N(CH₃)₂, —NH(CH₃)₂⁺, —N(CH₃)₃⁺, —NHCH₂CH₃, —NH₂CH₂CH₃⁺, —N(CH₃)CH₂CH₃, —N(CH₂CH₃)₂, and —NH(CH₃)CH₂CH₃⁺. Alkylamino encompasses heteroalkyls containing one or two nitrogen atoms and one or more heteroatoms independently selected from the group consisting of oxygen and sulfur. The term alkylamino also contemplates heterocycloalkyl groups containing one or two nitrogen atoms, for example, a group NX¹X²X³, in which X¹ is H or a valency (e.g., X¹ is H), and X² and X³, together with the atom to which they are attached, are a 4- to 8-member ring that may contain valency, provided that NX¹X²X³ contains one valency. These include 4-member rings containing one nitrogen (azetidinyl), 5-member rings containing one nitrogen (e.g., pyrrolidinyl), or 6-member ring containing one nitrogen (e.g., piperidinyl). The cyclic alkylamino structures also include ring systems containing two nitrogen atoms, as well as substituted cyclic alkylamino structures, e.g., NX¹X²X³, where X¹ is alkyl, and X² and X³, together with the atom to which they are attached, are a 4- to 8-member ring that contains valency. Alkylamino is further exemplified by 3-azetidinyl that may be substituted or unsubstituted as described herein.

“Alkynyl,” as used herein, refers to a branched or unbranched, acyclic hydrocarbon group containing one or two carbon-carbon triple bonds. Alkynyl contains from 2 to 24 carbon atoms. Preferably, alkynyl is lower alkynyl. A lower alkynyl group contains from 2 to 6 carbon atoms (C₂₋₆alkynyl). Preferably, alkynyl is acyclic alkynyl (e.g., acyclic lower alkynyl). Alkynyl may be optionally substituted as described herein.

“Amide,” as used herein, refers to a group with the structure —CONX¹X², where X¹ and X² are independently H or an organic group such as an alkyl or aryl group.

“Amino acid,” as used herein, refers to a compound of formula NH(R¹)—CH(R²)—COOH or to a group —NR¹—CH(R²)—COOH, where R¹ is H and R² is optionally substituted alkyl, or R¹ and R², together with the atom to which each is attached, combine to form an optionally substituted heterocyclyl. In some embodiments, amino acid is a proteinogenic amino acid. Proteinogenic amino acids are known in the art. For example, proteinogenic amino acids are alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, and pyrrolysine. An ester of an amino acid is a compound of formula NH(R¹)—CH(R²)—COOR³ or to a group —NR¹—CH(R²)—COOR³, where each or R¹ and R² are as defined above, and R³ is optionally substituted alkyl.

“Aryl,” as used herein, refers to an aromatic carbocyclic or heterocyclic group having at least five atoms in a cyclic array. A carbocylic aryl can be a single 6- to 10-member ring (monocyclic) or a group of 2 or 3 fused rings (polycyclic), each ring independently being a 6- to 10-member ring. A heterocyclic aryl is called “heteroaryl.” Heteroaryl is a single 5- or 6-member ring (monocyclic) or a group of 2 or 3 fused rings (polycyclic), each ring independently being a 5- to 8-member ring, at least one of the rings containing at least one heteroatom that is oxygen, nitrogen, sulfur, or phosphorus. The ring(s) of heteroaryl contain at least one (e.g., from 1 to 4) heteroatom that is oxygen, nitrogen, sulfur, or phosphorus. Non-limiting examples of carbocyclic aryls include naphthalenyl and phenyl. Aryl may be unsubstituted or substituted as described herein.

“Cycloalkenyl,” as used herein, refers to a non-aromatic carbocyclic group having at least five atoms in a cyclic array and one or two endocyclic carbon-carbon double bonds. The cyclic array may be a 5- to 8-member ring (C5-C8 cycloalkenyl). Non-limiting examples of cycloalkyl groups include cyclopentenyl and cyclohexenyl.

“Cycloalkyl,” as used herein, refers to a non-aromatic carbocyclic or heterocyclic group having at least three atoms in a cyclic array. The cyclic array may be a 3- to 8-member ring. Non-limiting examples of cycloalkyl groups include cyclopropyl, cyclobutyl, cyclopentyl, and cyclohexyl. Heterocyclic cycloalkyl is called herein “heterocycloalkyl.” A heterocycloalkyl containing at least one endocyclic nitrogen is also termed a cycloalkylamino herein.

“Effective amount” is a quantity of a therapeutic agent (e.g., sobetirome, sobetirome prodrug, or a PPAR activator) sufficient to achieve a desired effect in a subject, or in a cell, being treated with the therapeutic agent. The effective amount of the therapeutic agent depends on several factors, including, but not limited to the subject or cells being treated, and the manner of administration of the therapeutic composition. In some embodiments, an “effective amount” of sobetirome, a sobetirome prodrug, or a PPAR activator, is the amount sufficient to promote myelination in a subject. In other embodiments, a “effective amount” of sobetirome, a sobetirome prodrug, or a PPAR activator, is the amount sufficient to reduce or inhibit demyelination in a subject.

“Ester,” as used herein, refers to a group with the structure —COOX where X is a substituent described herein. For example an ethyl ester has the structure —COOCH₂CH₃.

“Halogen” or “halide,” as used interchangeably herein, refer to F, Cl, Br, or I.

“Heteroalkyl,” as used herein, refers to (i) an acyclic alkyl, in which one, two, three, or four carbon atoms are replaced with heteroatom(s), each heteroatom independently selected from oxygen, nitrogen, sulfur, and phosphorus, or (ii) heterocycloalkyl. Heteroalkyl may be unsubstituted or substituted as described herein.

“Heterocyclyl,” as used herein, refers to an aromatic or non-aromatic cyclic group having at least three atoms in a cyclic array, at least one of the atoms (e.g., from 1 to 4) within the cyclic array being a heteroatom that is oxygen, nitrogen, sulfur, or phosphorus. The cyclic array may be a single 3- to 8-member ring (monocyclic) or a group of 2 or 3 fused rings (polycyclic), each ring independently being a 3- to 8-member ring. An aromatic heterocyclyl group is called “heteroaryl.” Heteroaryl is a single 5- or 6-member ring (monocyclic) or a group of 2 or 3 fused rings (polycyclic), each ring independently being a 5- to 8-member ring, at least one of the rings containing at least one heteroatom that is oxygen, nitrogen, sulfur, or phosphorus. The ring(s) of heteroaryl contain at least one (e.g., from 1 to 4) heteroatom that is oxygen, nitrogen, sulfur, or phosphorus. Non-aromatic heterocyclyl group is called “heterocycloalkyl.” Non-limiting examples of heterocyclyls include azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, oxetanyl, oxadiazolyl, oxiranyl, oxazinyl, oxazolyl, piperazinyl, pyrazinyl, pyridazinyl, pyrimidinyl, piperidinyl, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl, furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl, and pyridopyrimidinyl groups. Heterocyclyl may be unsubstituted or substituted as described herein.

A “pharmaceutically acceptable salt” refers to a salt of a therapeutic agent (e.g., sobetirome, a sobetirome prodrug, or a PPAR activator) which is, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free carboxylic acid group with a suitable base. Representative alkali or alkaline earth metal salts include sodium, lithium, potassium, calcium, magnesium, and the like, as well as nontoxic ammonium, primary ammonium, secondary ammonium, tertiary ammonium, or quaternary ammonium cations, including, but not limited to ammonium, tetramethylammonium, tetraethylammonium, methylammonium, dimethylammonium, trimethylammonium, triethylammonium, ethylammonium, and the like.

A “pharmaceutically acceptable excipient” is any ingredient other than sobetirome, a sobetirome prodrug, or a PPAR activator (e.g., a vehicle capable of suspending or dissolving the active compound). For example, a pharmaceutically acceptable excipient may be a vehicle capable of suspending or dissolving the active compound and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. The pharmaceutically acceptable excipients or carriers useful for each specific mode of administration are described herein below.

“PPAR activator,” as used herein, is a compound that activates peroxisome proliferator-activated receptor (PPAR) function in a cell upon being contacted with the cell or after being administered to an organism containing the cell. A PPAR activator may be a PPAR agonist, if the PPAR activator interacts with PPAR directly. PPAR activators may target only one PPAR (e.g., PPARγ agonists) or they may activate multiple PPARs (e.g., dual PPARα and PPARγ agonists). Non-limiting examples of PPAR agonists include drug classes: glitazones and glitazars. Non-limiting examples of glitazones known in the art include pioglitazone, rosiglitazone, lobeglitazone, ciglitazone, darglitazone, englitazone, netoglitazone, rivoglitazone, and 5-[4-[2-(5-(1-hydroxyethyl)-2-pyridinyl)ethoxy]benzyl]-2,4-thiazolidinedione. Non-limiting examples of glitazars known in the art include aleglitazar, muraglitazar, tesaglitazar, or saroglitazar.

Preventing or treating a disease: “Preventing,” as used herein in reference to a disease or condition, refers to a prophylactic treatment or treatment that prevents one or more symptoms or conditions of a disease, disorder, or conditions described herein. Preventive treatment that includes administration of sobetirome or a sobetirome prodrug and one or more PPAR activators can be chronic. The doses administered may be varied during the course of preventative treatment. “Treating,” as used herein in reference to a disease or condition, refers to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment.

“Sobetirome,” as used herein, refers to a compound of formula:

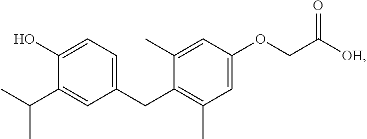 [SMILES: Cc1cc(OCC(=O)O)cc(C)c1Cc1ccc(O)c(C(C)C)c1]

or a pharmaceutically acceptable salt thereof.

“Sobetirome prodrug,” as used herein, refers to a sobetirome ester prodrug or sobetirome amide prodrug.

“Sobetirome ester prodrug” is a compound of formula:

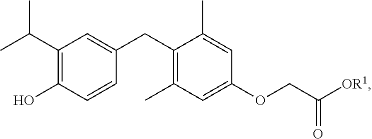

or a pharmaceutically acceptable salt thereof,

where

R¹ is unsubstituted alkyl, substituted alkyl, unsubstituted heteroalkyl, substituted heteroalkyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. In further embodiments, R¹ is alkyl or aryl. In yet further embodiments, R¹ can be ethyl, 2-trimethylaminoethyl, (N-morpholinyl)ethyl, 2-(lysinoyl)aminoethyl, 2-(valinoyl)aminoethyl, 2-(phenylalaninoyl)aminoethyl, or glucosyl. In still further embodiments, R¹ can be alkylamino, such as substituted alkylamino, cycloalkylamino or substituted cycloalkylamino. In additional examples, R¹ can be ethylamino, ethyl(N,N,N)-trimethylamino, ethylmorpholinyl, ethyl(N,N)-dimethylamino, 3-(N-methyl)azetidinyl, 4-pyrrolidinyl, 3-pyrrolidinyl, 2,2-dimethylethylamino, 3-(3-trifluoromethyl)azetidinyl, 2-pyrrolidinyl, 2-methylethylamino, 2-trifluoromethylamino, and N-methylethylamino. In some embodiments, R¹ is:

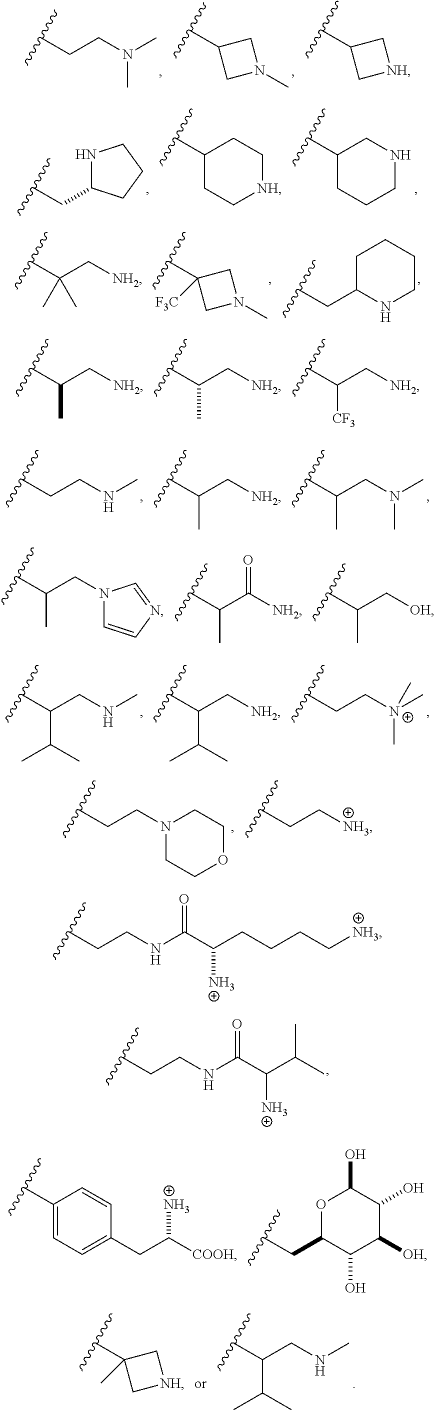

“Sobetirome amide prodrug” is a compound of formula:

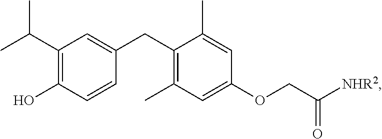

or a pharmaceutically acceptable salt thereof,

where

R² is optionally substituted alkyl or optionally substituted aryl. In some embodiments, R² is together with NH, to which it is attached, forms an amino acid or an ester thereof, where the NH group is α-amino group of the amino acid. In particular embodiments, R² is:

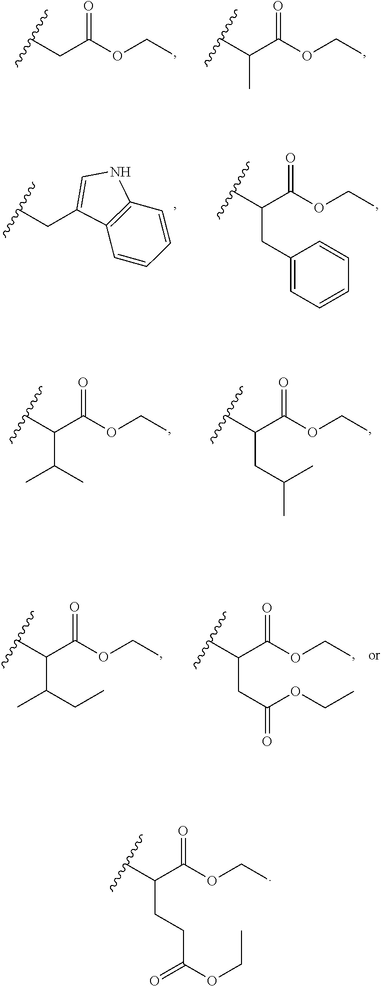

“Subject,” as used herein, refers to an animal (e.g., a mammal, such as a human). A subject to be treated according to the methods described herein may be one who has been diagnosed with a neurodegenerative disease involving demyelination, insufficient myelination, or under-development of a myelin sheath, or one at risk of developing the condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

Where a group is substituted, the group is substituted with 1, 2, 3, 4, 5, or 6 substituents, as valency permits. Each optional substituents is independently: C1-C6 alkyl; C2-C6 alkenyl; C2-C6 alkynyl; C3-C8 cycloalkyl; C5-C8 cycloalkenyl; three- to eight-membered heterocyclyl; C6-C10 aryl; five- to six-membered heteroaryl; halogen; azido (—N₃); nitro (—NO₂); cyano (—CN); acyloxy (—OC(═O)R′); acyl (—C(═O)R′); alkoxy (—OR′); amido; amino (—NRR′); carboxylic acid (—CO₂H), carboxylic ester (—CO₂R′); carbamoyl (—OC(═O)NR′R″ or —NRC(═O)OR′); hydroxy (—OH); oxo (═O); isocyano (—NC); sulfonate (—S(═O)₂OR); sulfonamide (—S(═O)₂NRR′ or —NRS(═O)₂R′); or sulfonyl (—S(═O)₂R), where each R, R′, and R″ is selected, independently, from H or an optionally substituted group that is C1-C6 alkyl, C2-C6 alkenyl, C2-C6 alkynyl, C3-C8 cycloalkyl, three- to eight-membered heterocyclyl, C6-C10 aryl, or five- to ten-membered heteroaryl. A substituted group may have, valency permitting, for example, 1, 2, 3, 4, 5, 6, 7, 8, or 9 substituents. In some embodiments, each hydrogen in a group may be replaced by a substituent group (e.g., perhaloalkyl groups such as —CF₃ or —CF₂CF₃ or perhaloaryls such as —C₆F₅). A substituent may itself be further substituted, valency permitting, with unsubstituted substituents defined herein. For example, a substituent may be substituted with 1, 2, 3, 4, 5, or 6 unsubstituted substituents as defined herein. For example, a lower C1-C6 alkyl or an aryl substituent group (e.g., heteroaryl, phenyl, or naphthyl) may be further substituted with 1, 2, 3, 4, 5, or 6 substituents as described herein, if valency permits.

### DETAILED DESCRIPTION

The invention provides methods of treating a demyelinating disease in a subject by administering sobetirome or a sobetirome prodrug and a peroxisome proliferator-activated receptor (PPAR) activator (e.g., PPAR agonist). Advantageously, administration of sobetirome or a sobetirome prodrug and a PPAR activator to a subject in accordance with the methods of the inventions can provide synergistically effective treatment or prevention of a demyelinating disease.

### Sobetirome

Sobetirome is a compound of formula:

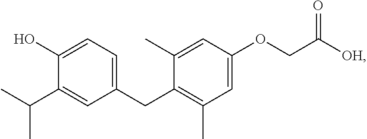 [SMILES: Cc1cc(OCC(=O)O)cc(C)c1Cc1ccc(O)c(C(C)C)c1]

or a pharmaceutically acceptable salt thereof.

Also disclosed are pharmaceutical compositions that include an effective amount of sobetirome and optionally one or more PPAR activators described herein.

The invention provides methods of treatment for demyelinating diseases that comprise administering sobetirome and one or more PPAR activators to a subject. The invention further provides compositions comprising sobetirome and one or more PPAR activators.

In some embodiments, sobetirome is administered at a dose of about 1 μg to about 500 μg. In some examples, the sobetirome or pharmaceutically acceptable salt thereof is administered at a dose of about 10 μg to about 100 μg.

In particular embodiments, the compound is administered to the subject once daily, twice daily, three times daily, once every two days, once weekly, twice weekly, three times weekly, once biweekly, once monthly, or once bimonthly. In some embodiments, sobetirome is administered daily (e.g., once daily or twice daily). In certain embodiments, sobetirome is administered to the subject once daily. In other embodiments, the effective amount is at least 30 μg (e.g., at least 50 μg, such as at least 100 m). In some embodiments, the effective amount is at least 30 μg (e.g., at least 50 μg, such as at least 100 μg) daily. In certain embodiments, the effective amount is at least 30 μg (e.g., at least 50 μg, such as at least 100 μg) twice daily. In particular embodiments, the effective amount is at least 30 μg (e.g., at least 50 μg, such as at least 100 μg) once weekly. In other embodiments, the effective amount is at least 30 μg (e.g., at least 50 μg, such as at least 100 μg) twice weekly. In certain embodiments, the effective amount is at least 30 μg (e.g., at least 50 μg, such as at least 100 μg) three times weekly. In some embodiments, the effective amount is 1 mg or less (e.g., 500 μg or less, e.g., 200 μg or less).

In some embodiments, a unit dosage form containing from 10 μg to 100 μg of sobetirome is administered once, twice, or three times per day. In some embodiments, a unit dosage form containing from 10 μg to 75 μg of sobetirome is administered once, twice, or three times per day. In other embodiments, a unit dosage form containing from 30 μg to 75 μg of sobetirome is administered once, twice, or three times per day. In particular embodiments, a unit dosage form containing from 10 μg to 50 μg of sobetirome is administered once, twice, or three times per day. In yet other embodiments, a unit dosage form containing from 30 μg to 50 μg of sobetirome is administered once, twice, or three times per day. In still other embodiments, a unit dosage form containing from 50 μg to 75 μg of sobetirome is administered once, twice, or three times per day.

### Sobetirome Prodrugs

Sobetirome prodrugs that may be used in the methods of the invention are sobetirome ester prodrugs or sobetirome amide prodrugs. The sobetirome ester prodrug is a compound of formula:

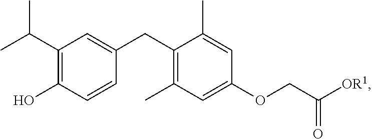

or a pharmaceutically acceptable salt thereof,

where

R¹ is unsubstituted alkyl, substituted alkyl, unsubstituted heteroalkyl, substituted heteroalkyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. In further embodiments, R¹ is alkyl or aryl. In yet further embodiments, R¹ can be ethyl, 2-trimethylaminoethyl, (N-morpholinyl)ethyl, 2-(lysinoyl)aminoethyl, 2-(valinoyl)aminoethyl, 2-(phenylalaninoyl)aminoethyl, or glucosyl. In still further embodiments, R¹ can be alkylamino, such as substituted alkylamino, cycloalkylamino or substituted cycloalkylamino. In additional examples, R¹ can be ethylamino, ethyl(N,N,N)-trimethylamino, ethylmorpholinyl, ethyl(N,N)-dimethylamino, 3-(N-methyl)azetidinyl, 4-pyrrolidinyl, 3-pyrrolidinyl, 2,2-dimethylethylamino, 3-(3-trifluoromethyl)azetidinyl, 2-pyrrolidinyl, 2-methylethylamino, 2-trifluoromethylamino, and N-methylethylamino.

In some embodiments, R¹ is:

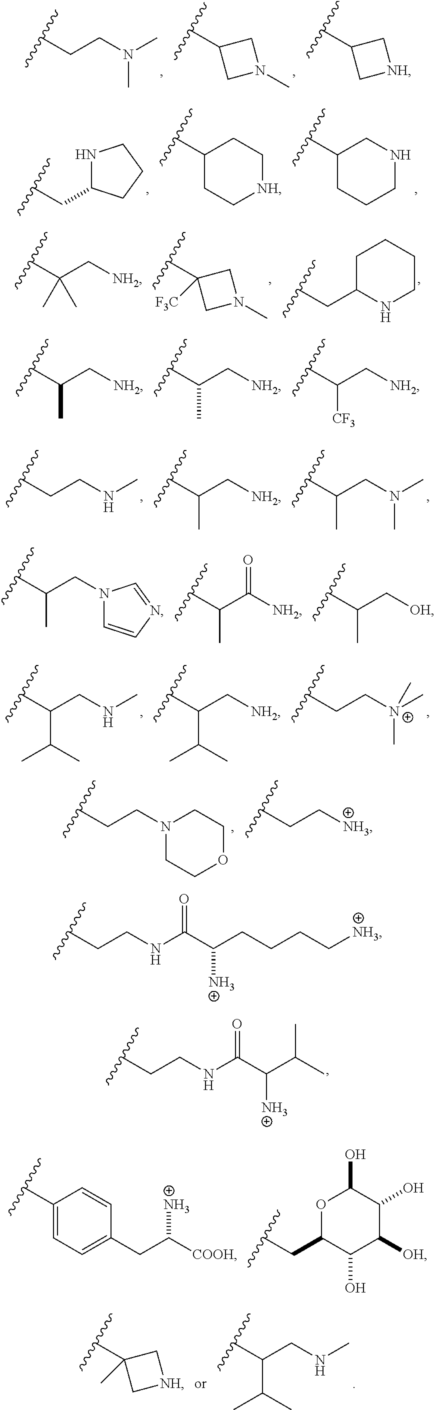

In some instances, the sobetirome prodrug is of the following formula:

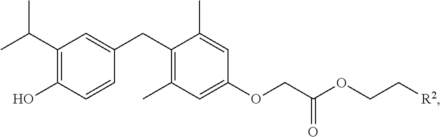

or a pharmaceutically acceptable salt thereof,

where R² is amino or alkylamino.

Examples of compounds of this structure include pharmaceutically acceptable salts (e.g., halide salts) of:

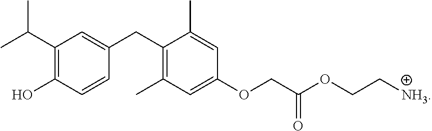

Another particular example is a compound of the following structure:

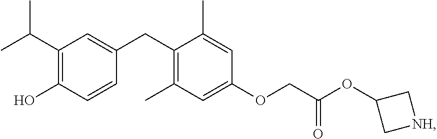 [SMILES: Cc1cc(OCC(=O)OC2CNC2)cc(C)c1Cc1ccc(O)c(C(C)C)c1]

or a pharmaceutically acceptable salt thereof.

A sobetirome amide prodrug is a compound of formula:

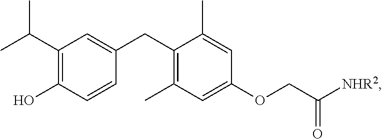

or a pharmaceutically acceptable salt thereof,

where

R² is optionally substituted alkyl or optionally substituted aryl. In some embodiments, R² is together with NH, to which it is attached, forms an amino acid or an ester thereof, where the NH group is α-amino group of the amino acid. In particular embodiments, R² is:

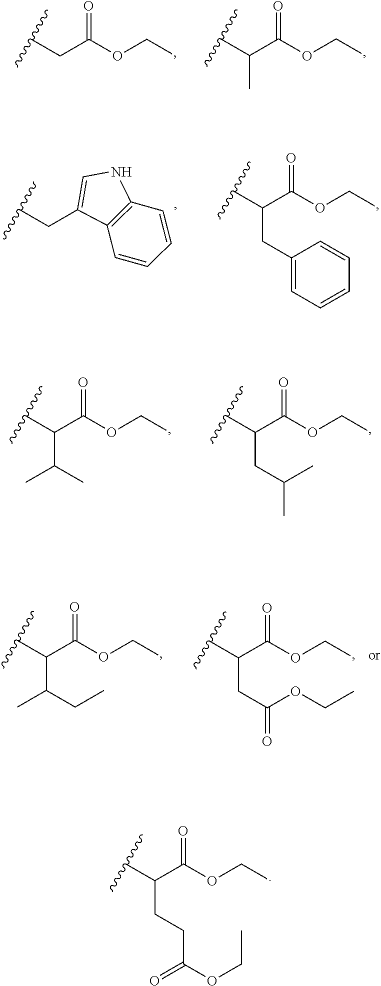

Sobetirome prodrugs can be prepared using methods known in the art. Non-limiting examples of sobetirome ester prodrugs and methods of their preparation are disclosed in US 2016/0244418. Sobetirome amide prodrugs may be prepared by subjecting sobetirome or an 0-protected version thereof to amidation reaction with an amine source. Typical amidation conditions include the use of reagents, such as EDC/DMAP, HATU/HOAt, or HBTU/HOAt. Alternatively, amidation conditions may involve Staudinger ligation (see, e.g., Kosal et al., Chem. Eur. J., 18:14444-14453, 2012; and Kosal et al., Angew. Chem. Int. Ed., 51, 12036-12040, 2012).

In some embodiments, a sobetirome prodrug is administered at a dose of about 1 μg to about 1 mg (e.g., to about 500 μg). In certain embodiments, a sobetirome prodrug is administered at a dose of about 10 μg to about 100 μg.

Also disclosed are pharmaceutical compositions that include an effective amount of a sobetirome prodrug and optionally one or more PPAR activators described herein.

In some embodiments, a sobetirome prodrug is administered at a dose of about 1 μg to about 500 μg. In certain embodiments, a sobetirome prodrug is administered at a dose of about 10 μg to about 100 μg.

In particular embodiments, the sobetirome prodrug is administered to the subject once daily, twice daily, three times daily, once every two days, once weekly, twice weekly, three times weekly, once biweekly, once monthly, or once bimonthly. In some embodiments, a sobetirome prodrug is administered daily (e.g., once daily or twice daily). In certain embodiments, a sobetirome prodrug is administered to the subject once daily. In other embodiments, the effective amount is at least 30 μg (e.g., at least 50 μg, e.g., at least 100 μg). In some embodiments, the effective amount is at least 30 μg (e.g., at least 50 μg, e.g., at least 100 μg) daily. In certain embodiments, the effective amount is at least 30 μg (e.g., at least 50 μg, e.g., at least 100 μg) twice daily. In particular embodiments, the effective amount is at least 30 μg (e.g., at least 50 μg, e.g., at least 100 μg) once weekly. In other embodiments, the effective amount is at least 30 μg (e.g., at least 50 μg, e.g., at least 100 μg) twice weekly. In certain embodiments, the effective amount is at least 30 μg (e.g., at least 50 μg, e.g., at least 100 μg) three times weekly. In some embodiments, the effective amount is 1 mg or less (e.g., 500 μg or less, e.g., 200 μg or less).

In some embodiments, a unit dosage form containing from 10 μg to 100 μg of a sobetirome prodrug is administered once, twice, or three times per day. In some embodiments, a unit dosage form containing from 10 μg to 75 μg of a sobetirome prodrug is administered once, twice, or three times per day. In other embodiments, a unit dosage form containing from 30 μg to 75 μg of a sobetirome prodrug is administered once, twice, or three times per day. In particular embodiments, a unit dosage form containing from 10 μg to 50 μg of a sobetirome prodrug is administered once, twice, or three times per day. In yet other embodiments, a unit dosage form containing from 30 μg to 50 μg of a sobetirome prodrug is administered once, twice, or three times per day. In still other embodiments, a unit dosage form containing from 50 μg to 75 μg of a sobetirome prodrug is administered once, twice, or three times per day.

### PPAR Activators

PPAR activators for use in the invention can be PPAR activators that interact with PPAR directly (e.g., PPAR agonists), including PPARγ agonists and dual PPARα and PPARγ agonists.

A PPARγ agonist for use in the methods and compositions of the invention can be pioglitazone, rosiglitazone, lobeglitazone, ciglitazone, darglitazone, englitazone, netoglitazone, rivoglitazone, or 5-[4-[2-(5-(1-hydroxyethyl)-2-pyridinyl)ethoxy]benzyl]-2,4-thiazolidinedione (disclosed in US 2016/0235729), or a pharmaceutically acceptable salt thereof. 5-[4-[2-(5-(1-hydroxyethyl)-2-pyridinyl)ethoxy]benzyl]-2,4-thiazolidinedione, or a pharmaceutically acceptable salt thereof, can be:
(R)-5-[4-[2-(5-((R)-1-hydroxyethyl)-2-pyridinyl)ethoxy]benzyl]-2,4-thiazolidinedione;
(R)-5-[4-[2-(5-((S)-1-hydroxyethyl)-2-pyridinyl)ethoxy]benzyl]-2,4-thiazolidinedione;
(S)-5-[4-[2-(5-((R)-1-hydroxyethyl)-2-pyridinyl)ethoxy]benzyl]-2,4-thiazolidinedione; or
(S)-5-[4-[2-(5-((S)-1-hydroxyethyl)-2-pyridinyl)ethoxy]benzyl]-2,4-thiazolidinedione,

or a pharmaceutically acceptable salt thereof.

A dual PPARα and PPARγ agonist for use in the methods and compositions of the invention can be aleglitazar, muraglitazar, tesaglitazar, or saroglitazar, or a pharmaceutically acceptable salt thereof.

The invention provides methods of treating demyelinating diseases by administering sobetirome or a sobetirome prodrug and one or more PPAR activators. In some embodiments, a single PPARγ agonist or dual PPARα and PPARγ agonist is administered. In some embodiments, one or more (e.g., 2, 3, or 4) PPARγ agonists are administered. In some embodiments, one or more (e.g., 2, 3, or 4) dual PPARα and PPARγ agonists are administered. In some embodiments, one or more (e.g., 2, 3, or 4) of PPARγ agonist or dual PPARα and PPARγ agonist are administered.

The invention also provides pharmaceutical compositions containing sobetirome or a sobetirome prodrug and one or more PPAR activators. In some embodiments, the pharmaceutical composition comprises a single PPARγ agonist or dual PPARα and PPARγ agonist. In some embodiments, the pharmaceutical composition comprises one or more (e.g., 2, 3, or 4) PPARγ agonists. In some embodiments, the pharmaceutical composition comprises one or more (e.g., 2, 3, or 4) dual PPARα and PPARγ agonists. In some embodiments, the pharmaceutical composition contains one or more (e.g., 2, 3, or 4) of PPARγ agonist or dual PPARα and PPARγ agonist.

### Demyelinating Diseases

Myelin is a lipid substance that forms a sheath around the axons of many nerve fibers. The myelin sheath is formed by oligodendrocytes during a process called myelination, and the sheath provides electrical insulation and speeds the conduction of nerve impulses in nerve fibers. Rapid conduction is important for proper nervous system function, and damage to the sheath (from, e.g., injury, toxin exposure, infection, inflammation, or disease) can be devastating. There is a need for treatments to prevent or reverse demyelination and/or promote myelination or remyelination, the repair or reformation of the myelin sheath.

The invention provides methods and compositions for treating demyelinating diseases. A demyelinating disease is any disease of the nervous system in which myelin is damaged or lost, or in which the growth or development of the myelin sheath is impaired. Demyelination inhibits the conduction of signals in the affected nerves, causing impairment in sensation, movement, cognition, or other functions for which nerves are involved. Demyelinating diseases have a number of different causes and can be hereditary or acquired. In some cases, a demyelinating disease is caused by an infectious agent, an autoimmune response, a toxic agent or traumatic injury. In other cases, demyelination is caused by ischemia or occurs during a neurodegenerative disease, such as Alzheimer's disease. In other cases, the cause of the demyelinating disease is unknown (“idiopathic”) or develops from a combination of factors.

The disease or condition to be treated by the methods or compositions of the invention can be any disease or condition associated with demyelination, insufficient myelination or underdevelopment of myelin sheath. In some embodiments, the disease or condition is multiple sclerosis, a leukodystrophy, a leukoencephalopathy, an idiopathic inflammatory demyelinating disease, or Alzheimer's disease.

In some embodiments, the disease is a leukoencephalopathy. Leukoencephalopathies are diseases affecting the white substance of the brain, for example, leukoencephalopathy with vanishing white matter and toxic leukoencephalopathy. Leukoencephalopathies are leukodystrophy-like diseases.

In some embodiments, the demyelinating disease is a leukodystrophy. Leukodystrophies are diseases that affect the growth or development of the myelin sheath. In some embodiments, the leukodystrophy is X-linked adrenoleukodystrophy (X-ALD, ALD, or X-linked ALD, also known as Addison-Schilder disease, Siemerling-Creutzfeldt disease, adrenomyeloneuropathy, or childhood cerenral adrenoleukodystrophy), Alexander disease, Pelizaeus-Merzbacher disease (PMD), Van der Knaap disease (also known as megalencephalic leukoencephalopathy with subcortical cysts (MLC)), Zellweger syndrome, Canavan disease (also known as Canavan-Van Bogaert-Bertrand disease, aspartoacylase deficiency and aminoacylase 2 deficiency), Cerebrotendineous xanthomatosis, Metachromatic leukodystrophy (MLD), or Krabbe disease (also known as globoid cell leukodystrophy or galactosylceramide lipidosis).

In some embodiments, the phenotype of X-linked adrenoleukodystrophy is childhood cerebral, adolescent, adrenomyeloneuropathy, adult cerebral, olivo-ponto-cerebellar, Addison disease, or asymptomatic. In other embodiments, the phenotype of X-linked adrenoleukodystrophy is asymptomatic, mild myelopathy, moderate to severe myelopathy (e.g., adrenomyeloneuropathy), cerebral, and adrenal. In certain embodiments, the phenotype of X-linked adrenoleukodystrophy is cerebral. In other embodiments, the phenotype of X-linked adrenoleukodystrophy is myelopathy (e.g., moderate to severe myelopathy). In certain other embodiments, the phenotype of X-linked adrenoleukodystrophy is asymptomatic. In yet other embodiments, the phenotype of X-linked adrenoleukodystrophy is Addison disease. In certain embodiments, the phenotype of X-linked adrenoleukodystrophy is olivo-ponto-cerebellar.

In some embodiments, the demyelinating disease is multiple sclerosis (MS). Multiple sclerosis is a slowly progressive CNS disease characterized by disseminated patches of demyelination in the brain and spinal cord, resulting in multiple and varied neurological symptoms and signs, usually with remissions and exacerbation. In some embodiments, the multiple sclerosis is relapsing-remitting multiple sclerosis (RRMS), secondary-progressive multiple sclerosis (SPMS), primary-progressive multiple sclerosis (PPMS), or progressive-relapsing multiple sclerosos (PRMS).

In some embodiments, the demyelinating disease is a neuropathy. A neuropathy is a functional disturbance or pathological change in the peripheral nervous system. An axonal neuropathy is a disorder that disrupts axonal function. In some embodiments, the neuropathy is paraproteinemic demyelinating polyneuropathy, chronic inflammatory demyelinating polyneuropathy (CIDP, also known as chronic relapsing polyneuropathy (CRP) or chronic inflammatory demyelinating polyradiculoneuropathy), Lewis-Sumner syndrome, Leber hereditary optic neuropathy, multifocal motor neuropathy (MMN), peroneal muscular atrophy (PMA, also known as Charcot-Marie-Tooth disease (CMT), Charcot-Marie-Tooth neuropathy and hereditary motor and sensory neuropathy (HMSN)), or Guillain-Barré syndrome. In some embodiments, the neuropathy is a subtype of Guillain-Barré syndrome, for example, acute inflammatory demyelinating polyneuropathy (AIDP), Miller Fischer syndrome, acute motor axonal neuropathy (Chinese paralytic syndrome), acute motor sensory axonal neuropathy, acute panautonomic neuropathy, or Bickerstaff's brainstem encephalitis. In some embodiments, the disease or condition is a chronic axonal neuropathy.

In some examples, the chronic inflammatory demyelinating polyneuropathy is multifocal acquired demyelinating sensory and motor neuropathy. In some examples, the chronic inflammatory demyelinating polyneuropathy is induced by HIV infection.

In some embodiments, the demyelinating disease is caused by a virus, infection, or immune deficiency, for example, progressive multifocal leukoencephalopathy (PML) or tropical spastic paraparesis (TSP, also known as HTLV-associated myelopathy or chronic progressive myelopathy)

In some embodiments, the demyelinating disorder is an idiopathic inflammatory demyelinating disease (IIDD). IIDDs include a broad spectrum of central nervous system disorders that can usually be differentiated on the basis of clinical, imaging, laboratory and pathological findings. Idiopathic inflammatory demyelinating diseases are sometimes known as borderline forms of multiple sclerosis. IIDD generally refers to a collection of multiple sclerosis variant diseases, including but not limited to, acute disseminated encephalomyelitis (ADEM, known as recurrent disseminated encephalomyelitis or multiphasic disseminated encephalomyelitis after more than one demyelinating episode), Balo concentric sclerosis, Diffuse myelinoclastic sclerosis (also known as Schilder's disease), Marburg multiple sclerosis (also known as tumefactive multiple sclerosis or fulminant multiple sclerosis), Devic's syndrome (also known as Devic's disease or neuromyelitis optica (NMO)), acute hemorrhagic leukoencephalitis (AHL or AHLE, also known as acute necrotizing encephalopathy (ANE), acute hemorrhagic encephalomyelitis (AHEM), acute necrotizing hemorrhagic leukoencephalitis (ANHLE), Weston-Hurst syndrome, or Hurst's disease), optic-spinal MS, transverse myelitis, and solitary sclerosis.

In some embodiments, the demyelinating disease is cerebral palsy.

In some embodiments, the disease or condition is central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barré syndrome, infantile Refsum disease, adult Refsum disease 1, adult Refsum disease 2, Zellweger syndrome, X-linked adrenoleukodystrophy (X-ALD), metachromatic leukodystrophy, Krabbe disease, Pelizaeus-Merzbacher disease, Canavan disease, Alexander disease, Binswanger's disease, peroneal muscular atrophy, cerebrotendineous xanthomatosis, leukoencephalopathy with vanishing white matter, toxic leukoencephalopathy, van der Knaap disease, progressive multifocal leukoencephalopathy, Marchiafava-Bignami disease or transverse myelitis.

In some embodiments, the disease or condition results from intraventricular hemorrhage, neonatal hypoxia, or acute hypoxemic respiratory failure.

In some embodiments of the disclosed method, administration of sobetirome or a sobetirome prodrug and one or more PPAR activators prevents or mitigates at least one symptom of the disease or condition. In some examples, the symptom is a lack of sphincter control, erectile dysfunction, paraparesis, ataxia, adrenocortical insufficiency, progressive neuropathy, paresthesia, dysarthria, dysphagia, clonus, or any combination thereof.

In some embodiments, administration of sobetirome, a sobetirome prodrug, and one or more PPAR activators prevents or mitigates damage to central nervous system myelin, peripheral nervous system myelin, adrenal cortex, testicular Leydig cells, or any combination thereof

### Therapeutic Methods

In some embodiments, sobetirome or a sobetirome prodrug is administered in combination with one or more PPAR activators. In one example, sobetirome or a sobetirome prodrug and one or more PPAR activators are administered together in the same pharmaceutical composition. In another example, sobetirome or a sobetirome prodrug and one or more PPAR activators are administered separately at about the same time (e.g., one minute apart or less, or five minutes apart or less). In some embodiments, sobetirome or a sobetirome prodrug and one or more PPAR activators are administered separately via the same route of administration (e.g., intravenous injection). In some embodiments, sobetirome or a sobetirome prodrug and one or more PPAR activators are administered separately via different routes of administration (e.g., intravenous injection of sobetirome or a sobetirome prodrug and oral administration of one or more PPAR activators).

In some embodiments, sobetirome or a sobetirome prodrug is administered prior to a one or more PPAR activators. In further embodiments, sobetirome or a sobetirome prodrug is administered within 1 hour of the one or more PPAR activators (e.g., before, e.g., 15 min, 30 min, or 1 hour before). In some embodiments, sobetirome or a sobetirome prodrug is administered within 12 hours of the one or more PPAR activators administration (e.g., before, e.g., 1, 2, 3, 4, 5, 6, 7, 8, 9, 10, 11, or 12 hours before). In certain embodiments, sobetirome or a sobetirome prodrug is administered within 24 hours of the one or more PPAR activators administration (e.g., before, e.g., 12 or 24 hours before). In particular embodiments, sobetirome or a sobetirome prodrug is administered within 1 week of the one or more PPAR activators administration (e.g., before, e.g., 1, 2, 3, 4, 5, or 6 days before). In some embodiments, sobetirome or a sobetirome prodrug is administered within 1 month of the one or more PPAR activators administration (e.g., before, e.g., 1, 2, 3, or 4 weeks before).

In some embodiments, sobetirome or a sobetirome prodrug is administered after a one or more PPAR activators. In further embodiments, sobetirome or a sobetirome prodrug is administered within 1 hour of the one or more PPAR activators (e.g., after, e.g., 15 min, 30 min, or 1 hour after). In some embodiments, sobetirome or a sobetirome prodrug is administered within 12 hours of the one or more PPAR activators administration (e.g., after, e.g., 1, 2, 3, 4, 5, 6, 7, 8, 9, 10, 11, or 12 hours after). In certain embodiments, sobetirome or a sobetirome prodrug is administered within 24 hours of the one or more PPAR activators administration (e.g., after, e.g., 12 or 24 hours after). In particular embodiments, sobetirome or a sobetirome prodrug is administered within 1 week of the one or more PPAR activators administration (e.g., after, e.g., 1, 2, 3, 4, 5, or 6 days after). In some embodiments, sobetirome or a sobetirome prodrug is administered within 1 month of the one or more PPAR activators administration (e.g., after, e.g., 1, 2, 3, or 4 weeks after).

In some embodiments, sobetirome or a sobetirome prodrug is administered at a dose of about 1 μg to about 1 mg (e.g., about 1 μg to about 500 μg). In some examples, sobetirome or a sobetirome prodrug is administered at a dose of about 10 μg to about 100 μg.

In some embodiments, sobetirome or a sobetirome prodrug is administered daily.

In some embodiments, sobetirome or a sobetirome prodrug is administered at a dose of about 1 μg to about 500 μg. In further embodiments, sobetirome or a sobetirome prodrug is administered at a dose of about 10 μg to about 100 μg. In yet further embodiments, sobetirome or a sobetirome prodrug is administered at a dose of about 10 μg to about 30 μg. In still further embodiments, sobetirome or a sobetirome prodrug is administered at a dose of about 30 μg to about 50 μg. In certain embodiments, sobetirome or a sobetirome prodrug is administered at a dose of about 50 μg to about 70 μg. In particular embodiments, sobetirome or a sobetirome prodrug is administered at a dose of about 70 μg to about 100 μg. In other embodiments, sobetirome or a sobetirome prodrug is administered at a dose of about 100 μg to about 200 μg. In yet other embodiments, sobetirome or a sobetirome prodrug is administered at a dose of about 200 μg to about 400 μg. In still other embodiments, sobetirome or a sobetirome prodrug is administered at a dose of about 400 μg to about 1 mg.

In some embodiments, sobetirome or a sobetirome prodrug is administered daily. In particular embodiments, sobetirome or a sobetirome prodrug is administered to the subject once daily, twice daily, three times daily, once every two days, once weekly, twice weekly, three times weekly, once biweekly, once monthly, or once bimonthly. In certain embodiments, sobetirome or a sobetirome prodrug is administered to the subject once daily. In other embodiments, the effective amount is at least 30 μg (e.g., at least 50 μg, such as at least 100 m). In some embodiments, the effective amount is at least 30 μg (e.g., at least 50 μg, such as at least 100 μg) daily. In certain embodiments, the effective amount is at least 30 μg (e.g., at least 50 μg, such as at least 100 μg) twice daily. In particular embodiments, the effective amount is at least 30 μg (e.g., at least 50 μg, such as at least 100 μg) once weekly. In other embodiments, the effective amount is at least 30 μg (e.g., at least 50 μg, such as at least 100 μg) twice weekly. In certain embodiments, the effective amount is at least 30 μg (e.g., at least 50 μg, such as at least 100 μg) three times weekly. In some embodiments, the effective amount is 1 mg or less (e.g., 500 μg or less, e.g., 200 μg or less).

In some embodiments, a unit dosage form containing from 10 μg to 100 μg of sobetirome or a sobetirome prodrug is administered once, twice, or three times per day. In some embodiments, a unit dosage form containing from 10 μg to 75 μg of sobetirome or a sobetirome prodrug is administered once, twice, or three times per day. In other embodiments, a unit dosage form containing from 30 μg to 75 μg of sobetirome or a sobetirome prodrug is administered once, twice, or three times per day. In particular embodiments, a unit dosage form containing from 10 μg to 50 μg of sobetirome or a sobetirome prodrug is administered once, twice, or three times per day. In yet other embodiments, a unit dosage form containing from 30 μg to 50 μg of sobetirome or a sobetirome prodrug is administered once, twice, or three times per day. In still other embodiments, a unit dosage form containing from 50 μg to 75 μg of sobetirome or a sobetirome prodrug is administered once, twice, or three times per day.

In some embodiments, about 0.1 mg to about 600 mg of one or more PPAR activators are administered. In some embodiments, about 2 mg to about 200 mg of one or more PPAR activators are administered.

In some embodiments, one or more PPAR activators are administered daily.

In particular embodiments, one or more PPAR activators are administered to the subject once daily, twice daily, three times daily, once every two days, once weekly, twice weekly, three times weekly, once biweekly, once monthly, or once bimonthly. In certain embodiments, one or more PPAR activators are administered to the subject once daily. In other embodiments, the effective amount is at least 2 mg (e.g., at least 10 mg, such as at least 30 mg). In some embodiments, the effective amount is at least 2 mg (e.g., at least 10 mg, such as at least 30 mg) daily. In certain embodiments, the effective amount is at least 2 mg (e.g., at least 10 mg, such as at least 30 mg) twice daily. In particular embodiments, the effective amount is at least 2 mg (e.g., at least 10 mg, such as at least 30 mg) once weekly. In other embodiments, the effective amount is at least 2 mg (e.g., at least 10 mg, such as at least 30 mg) twice weekly. In certain embodiments, the effective amount is at least 2 mg (e.g., at least 10 mg, such as at least 30 mg) three times weekly. In some embodiments, the effective amount is 600 mg or less (e.g., 200 mg or less, such as 50 mg or less).

In some embodiments, the methods of the present disclosure involve administering a unit dosage form containing from 2 mg to 200 mg of a PPAR activator, once, twice or three times per day. In some embodiments, the methods of the present disclosure involve administering a unit dosage form containing from 2 mg to 100 mg of a PPAR activator, once, twice or three times per day. In other embodiments, the methods of the present disclosure involve administering a unit dosage form containing from 4 mg to 100 mg of a PPAR activator, once, twice or three times per day. In particular embodiments, the methods of the present disclosure involve administering a unit dosage form containing from 2 mg to 50 mg of a PPAR activator, once, twice or three times per day. In yet other embodiments, the methods of the present disclosure involve administering a unit dosage form containing from 4 mg to 50 mg of a PPAR activator, once, twice or three times per day. In still other embodiments, the methods of the present disclosure involve administering a unit dosage form containing from 50 mg to 100 mg of a PPAR activator, once, twice or three times per day.

Sobetirome, sobetirome prodrugs, and PPAR activators can be administered using any suitable route of administration for the treatment of a disease or condition associated with demyelination, insufficient myelination, or underdevelopment of myelin sheath. For example, standard routes of administration include oral, parenteral, or topical routes of administration. In particular, the route of administration of sobetirome, a sobetirome prodrug, and one or more PPAR activators may be oral. Parenteral routes of administration of sobetirome, a sobetirome prodrug, and one or more PPAR activators may be, e.g., buccal, sublingual, sublabial, by inhalation, intra-arterial, intravenous, intraventricular, intramuscular, subcutaneous, intraspinal, intraorbital, intrathecal, or intracranial. Topical routes of administration may be, e.g., cutaneous, intranasal, or ophthalmic.

### Pharmaceutical Compositions

The compounds used in the methods described herein are preferably formulated into pharmaceutical compositions for administration to human subjects in a biologically compatible form suitable for administration in vivo. Pharmaceutical compositions typically include one or more compounds as described herein and a pharmaceutically acceptable excipient.

The compounds used in the methods described herein can also be used in the form of salts, or as prodrugs, or pharmaceutical compositions thereof. All forms are within the scope of the invention. The compounds, salts, prodrugs, or pharmaceutical compositions thereof, may be administered to a subject in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, the compounds used in the methods described herein can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the compounds used in the methods described herein into preparations that can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. The active compound can be milled to a particle size of 200 mesh or less (e.g., about 40 mesh).

### Formulations

A compound identified as capable of treating any of the conditions described herein, using any of the methods described herein, may be administered to subjects or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer one or more compounds used in the methods described herein to subjects suffering from a disease in which demyelination occurs. Administration may begin before the subject is symptomatic.

Exemplary routes of administration of compounds described herein, or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. Compounds used in the methods described herein desirably are administered with a pharmaceutically acceptable carrier. Pharmaceutical formulations of compounds formulated for treatment of the disorders described herein are also part of the present invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Buccal Administration

Dosages for buccal or sublingual administration typically may contain up to 500 mg of an active agent per single dose as required. In practice, the physician determines the actual dosing regimen which is most suitable for an individual subject, and the dosage varies with the age, weight, and response of the particular subject. The above dosages are exemplary of the average case, but individual instances exist wherein higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a compound of the invention with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of compounds of the invention. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., Biesalski, U.S. Pat. No. 5,112,598 and Biesalski, U.S. Pat. No. 5,556,611, each of which is herein incorporated by reference).

### Formulations for Nasal or Inhalation Administration

The compounds may also be formulated for nasal administration. Compositions for nasal administration also may conveniently be formulated as aerosols, drops, gels, and powders. The formulations may be provided in a single or multidose form. In the case of a dropper or pipette, dosing may be achieved by the subject administering an appropriate, predetermined volume of the solution or suspension. In the case of a spray, this may be achieved, for example, by means of a metering atomizing spray pump.

The compounds may further be formulated for aerosol administration, particularly to the respiratory tract by inhalation and including intranasal administration. The compound will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the compound in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form comprises an aerosol dispenser, it will contain a propellant, which can be a compressed gas, e.g., compressed air or an organic propellant, e.g., fluorochlorohydrocarbon. The aerosol dosage forms can also take the form of a pump-atomizer.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection”: a liquid preparation that is a drug substance (e.g., sobetirome, a sobetirome prodrug, and/or one or more PPAR activators), or a solution thereof;
(2) “Drug for Injection”: the drug substance (e.g., sobetirome, a sobetirome prodrug, and/or one or more PPAR activators) as a dry solid that will be combined with the appropriate sterile vehicle for parenteral administration as a drug injection;
(3) “Drug Injectable Emulsion”: a liquid preparation of the drug substance (e.g., sobetirome, a sobetirome prodrug, and/or one or more PPAR activators) that is dissolved or dispersed in a suitable emulsion medium;
(4) “Drug Injectable Suspension”: a liquid preparation of the drug substance (e.g., sobetirome, a sobetirome prodrug, and/or one or more PPAR activators) suspended in a suitable liquid medium; and
(5) “Drug for Injectable Suspension”: the drug substance (e.g., sobetirome, a sobetirome prodrug, and/or one or more PPAR activators) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

The following examples are meant to illustrate the invention. They are not meant to limit the invention in any way.

### EXAMPLES

### Example 1. Evaluation of the Activity of a PPAR Activator In Vitro

### Evaluation of PPAR Activator Activity in Wild-Type Cells

A PPAR activator is added at varying concentrations to the culture medium of cultured cells expressing a PPAR-γ gene (e.g., COS cells, CV-1 cells, oligodendrocyte progenitor cells). Gene expression is then measured by microarray analysis, Northern blot analysis, or RT-PCR, using any appropriate fragment prepared from the PPAR-γ nucleic acid molecule as a hybridization probe. Protein production is measured using standard immunological techniques, such as Western blot analysis, immunoprecipitation, ELISA, or immunohistochemistry. The level of PPAR-γ gene expression or protein production in the presence of the PPAR activator is compared to the level measured in a control culture medium lacking the PPAR activator. A PPAR activator that promotes an increase in the expression of a PPAR-γ gene or the PPAR-γ protein level is considered useful in the invention and may be used as a therapeutic to treat a human subject suffering from a demyelinating disease. The same approach may be used to examine the effect of a PPAR activator on PPAR-α gene expression and protein production.

### Evaluation of PPAR Activator Activity Using Reporter Gene Assays

Direct Activation of Reporter Genes

The PPAR-γ transcriptional control region is cloned upstream from a luciferase reporter gene within a reporter vector. This vector is introduced into cells (e.g., COS cells, CV-1 cells, oligodendrocyte progenitor cells) along with an internal control reporter vector (e.g., a lacZ gene under the transcriptional regulation of the (3-actin promoter). A PPAR activator is then added at varying concentrations to the culture medium of cultured cells expressing the reporter vectors. After the cells are exposed to the PPAR activator, reporter gene activity is measured and PPAR-γ reporter gene activity is normalized to internal control reporter gene activity. A PPAR activator that promotes an increase in PPAR-γ reporter gene activity relative to internal control reporter activity may be used in the methods of the invention. This approach can be modified to evaluate effects on PPAR-α by cloning the PPAR-α transcriptional control region upstream of a reporter gene.

Transactivation of Reporter Genes

CV-1 cells are maintained in DME High Glucose medium (Irvine Scientific) supplemented with 10% fetal bovine serum and 2 mM Glutamine. Cells are split into D-MEM/F-12 medium (Gibco) supplemented with 10% charcoal-stripped fetal bovine serum for 3 days before harvesting. Cells are harvested into D-MEM/F-12 medium (Gibco) supplemented with 10% charcoal-stripped fetal bovine serum and counted. Cells are seeded at a density of 24,000 cells per well into 96-well plates and incubated overnight at 5% CO₂ and 37° C. Cells are transfected for 6 to 20 hours based on the Lipofectamine protocol (Gibco) with the following amounts of DNA per well: 2 ng PSG5 GAL4-human PPAR-γ, 8 ng UAS-tk-SPAP, 25 ng beta-gal, 45 ng pBluescript. See, e.g., Lehmann, J. M. et al., J. Biol. Chem., (1995), Vol. 270, pp 12953-12956 and. Brown, P. J. et al., Chem. Biol., (1997), Vol. 4, pp 909-918. Cells are then incubated overnight at 5% CO2 and 37° C. PPAR activators are solublized to 10 mM in DMSO and then serially diluted from 1e-5 M to 1e-10 M into D-MEM/F-12 (Gibco) medium supplemented with 10% delipidated and charcoal-stripped heat inactivated calf serum (Sigma), 2 mM Glutamine, and Pen-Strep. PPAR activator dilutions are added at 100 μl/well to the transfected cell plates after aspiration of the transfection media. DMSO controls and 1 μM rosiglitazone controls are added to each cell plate. Cells are incubated overnight at 5% CO₂ and 37° C. and then lysed with 2.5 μl 0.5% Triton X-100. Two daughter plates are made from each mother plate. One daughter receives 200 μl/well SPAP substrate (Sigma 1.04) and the other daughter receives 200 μl/well beta-gal substrate (Sigma N-1127). Once developed, cell plates are read at 405 nM. SPAP data are normalized to beta-gal, and % transactivation is calculated relative to the DMSO negative control. A PPAR-α cell-based reporter assay may be performed as described for PPAR-γ if GAL4-human PPAR-α is used in place of GAL4-human PPAR-γ and 2-(4-(2-(1-Heptyl-3-(4-fluorophenyl)ureido)ethyl)phenoxy)-2-methylpropionic acid is used in place of rosiglitazone.

### Example 2. Treatment of a Demyelinating Disease In Vivo by Administration of Sobetirome and a PPAR Activator

Effect of Sobetirome and a PPAR Activator on Demyelination in the Lysolecithin Toxin Model of Focal Demyelination

Lysolecithin is injected into the corpus callosum of C57BL/6 mice using stereotactic equipment at the x, y, z coordinates of +1.000, +1.050, and +2.000 mm from the Bregma point using a beveled needle with the bevel facing caudally. To minimize trauma, 2 μL of 2% lysolecithin or 2 μL of PBS is injected over four minutes using a micropump injector and the beveled needle is held in place for five minutes before withdrawal. Brains are harvested 8 days later and fixed in paraformaldehyde. Free-floating slices of 30 μm are sectioned with a vibratome and stained with BlackGold® to detect myelin. In lysolecithin-injected (but not PBS-injected) C57BL/6 mice, demyelination is observed in 6-8 serial sections in the corpus callosum.

To determine how administration of sobetirome and a PPAR activator initiated before lysolecithin injection affects the extent of demyelination 8 days after lysolecithin injection, sobetirome is administered daily at 0.1-20 mg/kg (e.g., 1, 5, 10, or 15 mg/kg/day) (e.g., by i.p. injection or by gavage). Mice are also administered with a PPAR activator (e.g., pioglitazone) at a dose of e.g., 1-40 mg/kg/day (e.g., 5, 10, 15, 20, 25, 30, or 35 mg/kg/day) starting before and for a period of time after lysolecithin injection. Control mice are daily administered vehicle by the same route of administration as sobetirome and/or a PPAR activator starting before and continuing for a predetermined period of time after lysolecithin injection.

Effect of Sobetirome and a PPAR Activator on the Demyelination in EAE Animal Model

Experimental autoimmune encephalomyelitis (EAE) is a useful and widely accepted model for studying mechanisms of CNS tissue injury and for testing potential therapies for MS. To determine the effect of sobetirome and a PPAR activator on EAE, EAE is induced in C57BL/6 female mice by immunization with MOG 35-55 peptide. Two to three weeks (e.g., 17 days) after immunization, at the peak of clinical disease, mice are randomized to receive different dosing regimens of sobetirome (e.g., 0.1-20 mg/kg/day, e.g., 1, 5, 10, or 15 mg/kg/day) and pioglitazone (1-40 mg/kg/day, e.g., 5, 10, 15, 20, 25, 30, or 35 mg/kg/day) or vehicle. After a predetermined period of time, mice are euthanized and processed for histologic examination.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a subject having or at risk of developing X-linked adrenoleukodystrophy, comprising administering to the subject an effective amount of sobetirome or a sobetirome prodrug and one or more PPAR activators.

2. A method of treating a subject having or at risk of developing a disease or condition associated with demyelination, insufficient myelination, or underdevelopment of myelin sheath, comprising administering to the subject an effective amount of sobetirome or a sobetirome prodrug and one or more PPAR activators.

3. The method of claim 2, wherein the disease or condition is multiple sclerosis, a leukodystrophy, a leukoencephalopathy, an idiopathic inflammatory demyelinating disease, or Alzheimer's disease.

4. The method of claim 3, wherein the multiple sclerosis is relapsing-remitting multiple sclerosis, primary-progressive multiple sclerosis, secondary-progressive multiple sclerosis, or progressive-relapsing multiple sclerosis.

5. The method of claim 2, wherein the disease or condition is central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barré syndrome, infantile Refsum disease, adult Refsum disease 1, adult Refsum disease 2, Zellweger syndrome, X-linked adrenoleukodystrophy (X-ALD), metachromatic leukodystrophy, Krabbe disease, Pelizaeus-Merzbacher disease, Canavan disease, Alexander disease, peroneal muscular atrophy, cerebrotendineous xanthomatosis, Binswanger's disease, leukoencephalopathy with vanishing white matter, toxic leukoencephalopathy, van der Knaap disease, progressive multifocal leukoencephalopathy, Marchiafava-Bignami disease or transverse myelitis.

6. The method of claim 5, wherein the Guillain-Barré syndrome is acute inflammatory demyelinating polyneuropathy.

7. The method of claim 5, wherein the chronic inflammatory demyelinating polyneuropathy is multifocal acquired demyelinating sensory and motor neuropathy.

8. The method of claim 5, wherein the chronic inflammatory demyelinating polyneuropathy is induced by HIV infection.

9. The method of claim 5, wherein the X-linked adrenoleukodystrophy is adrenomyeloneuropathy or childhood cerebral adrenoleukodystrophy.

10. The method of claim 5, wherein the X-linked adrenoleukodystrophy is Addison's disease.

11. The method of claim 2, wherein the disease or condition is a chronic axonal neuropathy.

12. The method of claim 2, wherein the disease or condition results from intraventricular hemorrhage, neonatal hypoxia, or acute hypoxemic respiratory failure.

13. The method of claim 2, wherein the disease or condition is cerebral palsy.

14-16. (canceled)

17. The method of claim 1, wherein each of the one or more PPAR activators is independently a PPARγ agonist or a dual PPARα and PPARγ agonist.

18. The method of claim 17, wherein the PPARγ agonist is pioglitazone, rosiglitazone, lobeglitazone, ciglitazone, darglitazone, englitazone, netoglitazone, rivoglitazone, or 5-[4-[2-(5-(1-hydroxyethyl)-2-pyridinyl)ethoxy]benzyl]-2,4-thiazolidinedione, or a pharmaceutically acceptable salt thereof.

19-27. (canceled)

28. The method of claim 17, wherein the dual PPARα and PPARγ agonist is aleglitazar, muraglitazar, tesaglitazar, or saroglitazar, or a pharmaceutically acceptable salt thereof.

29-71. (canceled)

72. A pharmaceutical composition comprising sobetirome or a sobetirome prodrug, one or more PPAR activators, and a pharmaceutically acceptable excipient.

73. The pharmaceutical composition of claim 72, wherein each of the one or more PPAR activators is independently a PPARγ agonist or a dual PPARα and PPARγ agonist.

74. The pharmaceutical composition of claim 73, wherein each of the one or more PPAR activators is the PPARγ agonist independently selected from the group consisting of pioglitazone, rosiglitazone, lobeglitazone, ciglitazone, darglitazone, englitazone, netoglitazone, rivoglitazone, and 5-[4-[2-(5-(1-hydroxyethyl)-2-pyridinyl)ethoxy]benzyl]-2,4-thiazolidinedione, and pharmaceutically acceptable salts thereof.

75-83. (canceled)

84. The pharmaceutical composition of claim 73, wherein each of the one or more PPAR activators is independently a dual PPARα and PPARγ agonist selected from the group consisting of aleglitazar, muraglitazar, tesaglitazar, and saroglitazar, and pharmaceutically acceptable salts thereof.

85-105. (canceled)
